(19) United States
(12) Patent Application Publication
(10) Pub. No.: US 2026/0199808 A1
(43) Pub. Date: Jul. 16, 2026

(54) CROSSLINKED STREPTAVIDIN CHROMATOGRAPHIC MATERIALS AND METHODS OF USE THEREOF

(71) Applicant: Waters Technologies Corporation, Milford, MA (US)

(72) Inventors: Beatrice Muriithi, Attleboro, MA (US); Yeliz Tunc Sarisozen, Westford, MA (US); Martin Gilar, Franklin, MA (US); Kevin Wyndham, Worcester, MA (US)

(73) Assignee: Waters Technologies Corporation, Milford, MA (US)

(21) Appl. No.: 19/448,715

(22) Filed: Jan. 14, 2026

Related U.S. Application Data
(60) Provisional application No. 63/744,962, filed on Jan. 14, 2025.

(51) Int. Cl. B01D 15/38; B01J 20/26; B01J 20/28; B01J 20/289; B01J 20/32; C07K 14/36

(52) U.S. Cl. CPC B01D 15/3823; B01J 20/267; B01J 20/28004; B01J 20/289; B01J 20/321; B01J 20/3219; B01J 20/3282; C07K 14/36

(57) ABSTRACT

Disclosed herein are chromatographic materials comprising crosslinked streptavidin molecules. The crosslinked streptavidin chromatographic materials may be used to prepare affinity chromatographic materials using biotinylated affinity agents, such as a biotinylated antibody, biotinylated antigen-binding fragment, or biotinylated oligonucleotide. Due to the crosslinked nature of the streptavidin molecules, the materials provided herein have reduced to no streptavidin leachate, resulting in improved performance.

### CROSS-REFERENCE TO RELATED APPLICATION

This application claims priority from and the benefit of U.S. Application No. 63/744,962 filed on Jan. 14, 2025. The entire contents of this application are incorporated herein by reference.

### FIELD OF INVENTION

The present disclosure relates generally to methods of preparing crosslinked streptavidin-conjugated chromatographic materials and methods of using said materials in affinity chromatography.

### BACKGROUND

Streptavidin is used in affinity chromatography applications due to its strong non-covalent bond with biotin and biotinylated molecules. Streptavidin occurs as a homotetramer with four binding pockets that bind to biotin (or a biotinylated molecule). The streptavidin tetramer is increasingly stabilized by binding of each of the four binding sites; however, in the absence of said binding, the streptavidin tetramer is susceptible to dissociation under harsh conditions such as high temperatures, strong denaturants, or low pH. This dissociation releases streptavidin monomers, streptavidin leachate, which can interfere with protein-protein interactions, data analysis and overall sample quality. Accordingly, there exists a need in the art for methods of reducing streptavidin leachate from materials for affinity chromatography.

### SUMMARY OF INVENTION

The present technology provides crosslinked streptavidin-conjugated chromatographic materials, such as particles, that result in reduced streptavidin leachate. Said materials may be used to generate materials for affinity chromatography by binding a biotinylated affinity agent to the crosslinked streptavidin-conjugated materials. Due to the reduced leachate, the materials and methods provided herein may be used for affinity chromatography with reduced or no interference of streptavidin leachate.

Accordingly, in one aspect, disclosed herein is a method of generating crosslinked streptavidin particles, the method comprising a) contacting a plurality of streptavidin molecules with a crosslinker to generate a plurality of crosslinked streptavidin molecules; and b) contacting the plurality of crosslinked streptavidin molecules with a plurality of nonporous particles to generate a plurality of crosslinked streptavidin-conjugated nonporous particles.

In another aspect, disclosed herein is a method of generating crosslinked streptavidin particles, the method comprising contacting a plurality of streptavidin-conjugated nonporous particles with a crosslinker to generate a plurality of crosslinked streptavidin-conjugated nonporous particles.

In some embodiments, the crosslinker is a homo-functional or a hetero-functional crosslinker. In some embodiments, the crosslinker is a homo-functional crosslinker that is ethylene glycol diglycidyl ether.

In some embodiments, each particle of the plurality of particles comprises a nonporous polymer core and a hydrophilic surface on an outer layer of the nonporous polymer core. In some embodiments, the particle has an average particle size between 1 μm and 10 μm. In some embodiments, the nonporous polymer core has a gradient composition. In some embodiments, the nonporous polymer core comprises divinylbenzene (80%) and polystyrene. In some embodiments, the hydrophilic surface is selected from the group consisting of: (3-glycidyloxypropyl)trimethoxysilane, (3-glycidyloxypropyl)triethoxysilane, polyacrylate, glycidol, glyceroltriglycidyl ether, and poly(methyl acrylate). In some embodiments, the plurality of crosslinked streptavidin molecules and/or plurality of streptavidin molecules are conjugated to the hydrophilic surface of the particle via an epoxy linker. In some embodiments, the epoxy linker has a formula of:

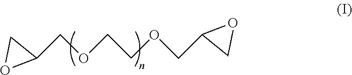

wherein n is between 1-12. In some embodiments, n is 1, 4, or 9. In some embodiments, n is 1. In some embodiments, the plurality of crosslinked streptavidin molecules and/or plurality of streptavidin molecules conjugated to the hydrophilic surface provides a surface coverage of 2-6 μg/mg particle.

In one aspect, disclosed herein is a chromatographic column comprising a column body formed of a metal or a metal alloy, the column body housing a plurality of crosslinked streptavidin-conjugated nonporous particles generated by the methods provided herein.

In some embodiments, at least a portion of an interior surface of the column body is coated with an alkylsilyl material. In some embodiments, the chromatographic column further comprises frits within the column body, wherein the frits are coated with the alkylsilyl material. In some embodiments, the alkylsilyl material is a hydrophilic, non-ionic layer of polyethylene glycol silane.

In some embodiments, the column has no detectable leachate of streptavidin as determined by UV absorbance. In some embodiments, the column has a leachate absorbance value of <10 mAU as measured by UV absorbance at 280 nm.

In some embodiments, the column has at least an 85% reduction in leachate absorbance as compared to a column that does not comprise crosslinked streptavidin-conjugated particles. In some embodiments, the leachate absorbance is determined at the 4th peak eluted from the column as measured by UV absorbance at 280 nm. In some embodiments, the column has at least a 90% reduction or at least a 95% reduction in leachate absorbance.

In one aspect, disclosed herein is a chromatography column comprising a column body formed of a metal or a metal alloy, the column body housing a plurality of crosslinked streptavidin-conjugated nonporous particles.

In some embodiments, each particle of the plurality of particles comprises a nonporous polymer core and a hydrophilic surface on an outer layer of the nonporous polymer core. In some embodiments, the particle has an average particle size between 1 μm and 10 μm. In some embodiments, the nonporous polymer core has a gradient composition. In some embodiments, the nonporous polymer core comprises divinylbenzene (80%) and polystyrene. In some embodiments, the hydrophilic surface is selected from the group consisting of: (3-glycidyloxypropyl)trimethoxysilane, (3-glycidyloxypropyl)triethoxysilane, polyacrylate, glycidol, glyceroltriglycidyl ether, and poly(methyl acrylate). In some embodiments, the plurality of crosslinked streptavidin molecules and/or plurality of streptavidin molecules are conjugated to the hydrophilic surface of the particle via an epoxy linker. In some embodiments, the epoxy linker has a formula of:

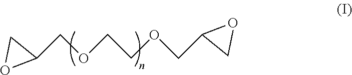

wherein n is between 1-12. In some embodiments, n is 1, 4, or 9. In some embodiments, n is 1. In some embodiments, the plurality of crosslinked streptavidin molecules and/or plurality of streptavidin molecules conjugated to the hydrophilic surface provides a surface coverage of 2-6 μg/mg particle.

In one aspect, disclosed herein is a method of preparing an affinity chromatographic column, the method comprising: i) providing a chromatographic column described herein, ii) washing the chromatographic column with a wash buffer, and iii) applying a solution comprising a biotinylated affinity agent to the chromatographic column such that the biotinylated affinity agent binds to an accessible binding site present in the plurality of crosslinked streptavidin molecules, thereby forming an affinity chromatographic column.

In some embodiments, the method further comprises monitoring the eluent during step ii) with a UV detector. In some embodiments, there is no detectable leachate of streptavidin as measured by UV absorbance during step ii). In some embodiments, the UV absorbance at 280 nm is <10 mAU during step ii). In some embodiments, there is an at least 85% reduction in leachate absorbance as compared to a chromatographic column that does not comprise crosslinked streptavidin-conjugated particles. In some embodiments, the leachate absorbance is determined at the 4^{th} peak eluted from the column as measured by UV absorbance at 280 nm. In some embodiments, the column has at least a 90% reduction or at least a 95% reduction in leachate absorbance.

In some embodiments, the method further comprises step iv) applying a solution containing a target analyte to the affinity chromatographic column. In some embodiments, the method further comprises step v) washing the affinity chromatographic column with an elution buffer such that the target analyte is eluted from the column.

In some embodiments, the biotinylated affinity agent is a biotinylated antibody or biotinylated antigen-binding fragment thereof, or a biotinylated oligonucleotide.

### BRIEF DESCRIPTION OF THE DRAWINGS

The technology will be more fully understood from the following detailed description taken in conjunction with the accompanying drawings, in which:

FIG. 1A-1B are graphical illustrations of methods of preparing crosslinked streptavidin-conjugated chromatographic materials. FIG. 1A depicts a method wherein the streptavidin is crosslinked prior to conjugation to the chromatographic material. FIG. 1B depicts a method wherein the streptavidin is crosslinked after conjugation to the chromatographic material.

FIG. 2A-2B depict the reduction in leachate observed with crosslinked streptavidin-conjugated particles. FIG. 2A shows a chromatogram of the eluate from particles including crosslinked streptavidin. FIG. 2B shows a chromatogram of the eluate from particles including streptavidin (non-crosslinked).

FIG. 3A shows a chromatogram for a crosslinked streptavidin molecule.

FIG. 3B shows a chromatogram for a streptavidin molecule which is endcapped with biotin.

FIG. 3C shows a chromatogram for free streptavidin.

FIG. 3D shows a chromatogram for a BEH 200 standard.

FIG. 3E shows a mass spectra of the eluent from a column including non-conjugated streptavidin particles. Masses corresponding to both the polymer particle and streptavidin monomers are observed.

FIG. 3F shows the total ion mass spectra (TIC MS) for the eluate from a column including non-conjugated streptavidin particles.

FIG. 3G shows the detection of ions having a mass of about 677.47 Da (indicative of the polymer) for the eluate from a column including non-conjugated streptavidin particles.

FIG. 3H shows the detection of ions having a mass of about 1328.03 Da (indicative of streptavidin monomers) for the eluate from a column including non-conjugated streptavidin particles.

FIG. 3I shows a mass spectra of the eluent from a column including conjugated streptavidin particles. Masses corresponding to the polymer particle are observed, but no signals corresponding to streptavidin monomers are observed.

FIG. 3J shows the TIC MS for the eluate from a column including conjugated streptavidin particles.

FIG. 3K shows a base peak intensity mass spectra (BPI MS) for the eluate from a column including conjugated streptavidin particles.

FIG. 3L shows the UV-Vis spectroscopy signal from 210 nm to 400 nm for the eluate from a column including conjugated streptavidin particles.

FIG. 3M shows the mass spectra of the hydrophilic section of the polymer particle. The mass spectra shows several distinct peaks characteristic of the polymer.

FIG. 3N shows the detection of ions having a mass of about 625.38 Da (indicative of the polymer) for the eluate from a column including non-conjugated streptavidin particles.

FIG. 3O shows the detection of ions having a mass of about 581.36 Da (indicative of the polymer) for the eluate from a column including non-conjugated streptavidin particles.

FIG. 3P shows the detection of ions having a mass of about 537.33 Da (indicative of the polymer) for the eluate from a column including non-conjugated streptavidin particles.

FIG. 3Q shows the detection of ions having a mass of about 493.31 Da (indicative of the polymer) for the eluate from a column including non-conjugated streptavidin particles.

FIG. 3R shows the detection of ions having a mass of about 449.28 Da (indicative of the polymer) for the eluate from a column including non-conjugated streptavidin particles.

FIG. 4 depicts the effect of crosslinker length (n=1, ethylene glycol diglycidyl ether; n=9 poly(ethylene glycol) diglycidyl ether; and n=22 poly(ethylene glycol) diglycidyl ether) on the properties of the resulting streptavidin, compared to free streptavidin (top) and a BEH 200 standard (bottom). The shortest crosslinker examined (n=1) exhibited the least peak broadening and was the least shifted compared to unbound streptavidin.

FIGS. 5A-5C depict the ability for biotin to bind to accessible binding sites in particles of the present disclosure. FIG. 5A shows the elution of D-biotin from a column including crosslinked streptavidin-conjugated particles. FIG. 5B shows the elution of D-biotin from a column including streptavidin-conjugated particles (no crosslinking). FIG. 5C provides a quantification of the total amount of coupled biotin in each experiment.

FIGS. 6A-6B depict the ability for a biotinylated anti-insulin antibody to bind to accessible binding sites in crosslinked streptavidin-conjugated particles and streptavidin-conjugated particles. FIG. 6A provides the amount coupled over time as measured by the number of injections. FIG. 6B provides a quantification of the total amount of coupled antibody.

FIGS. 7A-7B depict the ability for a biotinylated anti-AAVX nanobody to bind to accessible binding sites in crosslinked streptavidin-conjugated particles (FIG. 7A) and streptavidin-conjugated particles (FIG. 7B).

### DETAILED DESCRIPTION

Disclosed herein are methods for preparing crosslinked streptavidin-conjugated chromatographic materials, such as particles and methods of use thereof. In order that the technology may be more readily understood, certain terms are first defined. In addition, it should be noted that whenever a value or range of values of a parameter are recited, it is intended that values and ranges intermediate to the recited values are also part of this disclosure. The word “about” if not otherwise defined means±5%. It is also to be noted that as used herein and in the claims, the singular forms “a,” “an,” and “the” include plural references unless the context clearly dictates otherwise.

### Definitions

As used herein, the term “conjugate” refers to a compound formed by the chemical bonding of a reactive functional group of one molecule, such as streptavidin, with an appropriately reactive functional group of another molecule, such as an epoxide. An example of suitably reactive functional groups is a nucleophile/electrophile pair. For instance, the nucleophile may be an amine group from an amino acid of streptavidin, and the electrophile is an epoxide.

As used herein, the term “conjugated” refers to the linkage of two molecules formed by the chemical bonding of a reactive functional group of one molecule, such as streptavidin, with an appropriately reactive functional group of another molecule, such as an epoxide.

As used herein, the term “crosslinked”, when used in reference to streptavidin, refers to a covalent bond formed between two streptavidin monomers via a crosslinker. As used herein, the term “crosslinker” refers to a compound having two reactive groups separated by a spacer arm. The two streptavidin monomers may be present within the same tetramer (intramolecular crosslinking) or between two tetramers (intermolecular crosslinking).

As used herein, the term “crosslinked streptavidin-conjugated particle” refers to a particle having one or more streptavidin tetramers conjugated to a surface of the particle, wherein one or more monomers present within the streptavidin tetramers are crosslinked.

As used herein, the term “crosslinked streptavidin-conjugated monoliths” refers to a monolith having one or more streptavidin tetramers conjugated to a surface of the monolith, wherein one or more monomers present within the streptavidin tetramers are crosslinked.

As used herein, the term “monolith” refers to a collection of individual particles packed into a bed formation, in which the shape and morphology of the individual particles are maintained. The particles are advantageously packed using a material that binds the particles together. Examples of suitable monoliths and binding materials are known in the art and further described in US Publication No. US 2023/0294073, incorporated herein by reference.

As used herein, the term “crosslinked streptavidin-conjugated membranes” refers to a membrane having one or more streptavidin tetramers conjugated to a surface of the membrane, wherein one or more monomers present within the streptavidin tetramers are crosslinked.

As used herein, the term “membrane” refers to a selective barrier, such as a semi-permeable barrier. A membrane may be a filtration membrane.

As used herein, the term “streptavidin leachate” refers to the dissociation of one or more streptavidin tetramers into streptavidin monomers, such that the monomers are no longer conjugated to a solid support, such as a nonporous particle described herein. Streptavidin leachate may be detected using known detection methods, including UV absorbance, and as described throughout the specification.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

### Methods of Preparing Crosslinked Streptavidin-Conjugated Materials

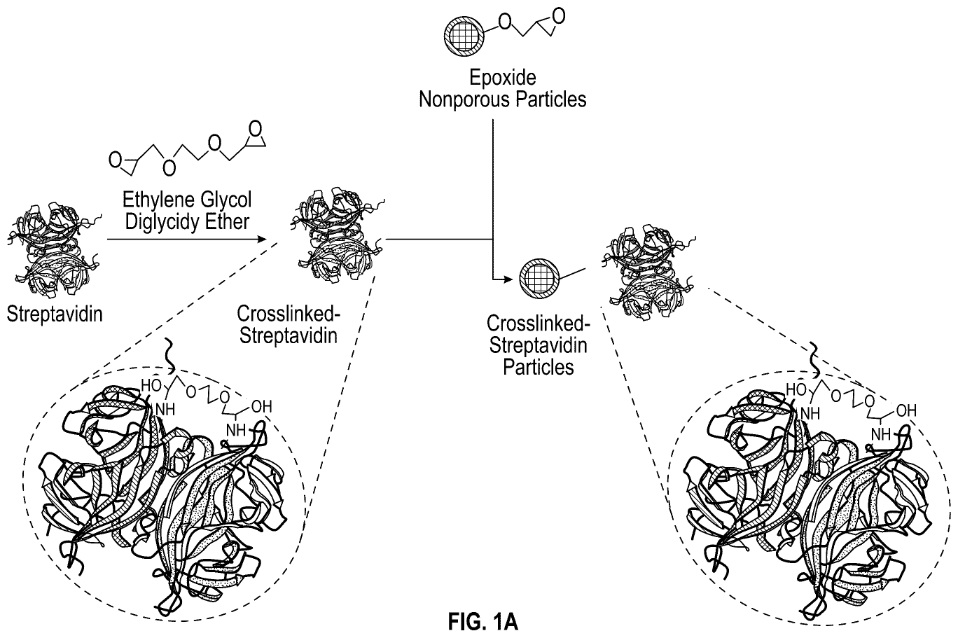

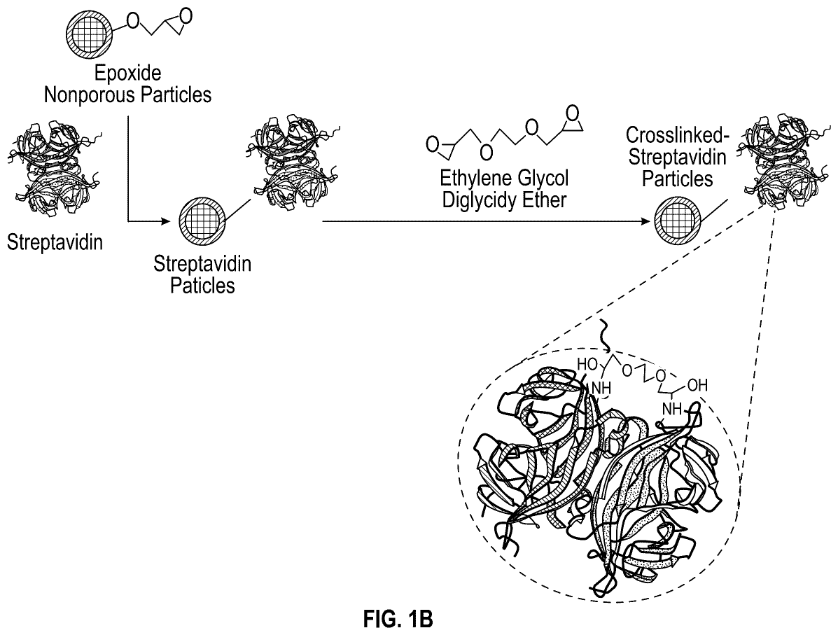

Provided herein are methods for preparing crosslinked streptavidin-conjugated chromatographic materials. One such method first utilizes a crosslinker to generate a plurality of crosslinked streptavidin molecules. The crosslinked streptavidin molecules may then be conjugated to the surface of a chromatographic material, such as a plurality of nonporous particles. For example, but not by way of limitation, nonporous particles may be prepared as described in Example 1. FIG. 1A graphically depicts said method wherein crosslinked streptavidin molecules are immobilized onto a plurality of nonporous particles. Alternatively, crosslinking of the streptavidin can occur after a plurality of streptavidin molecules (not crosslinked) are conjugated to the surface of a plurality of nonporous particles. FIG. 1B graphically depicts said method. Example 2 describes a method of preparing the crosslinked streptavidin-conjugated particles according to the method as shown in FIG. 1A. Example 3 describes a method of preparing the crosslinked streptavidin-conjugated particles according to the method as shown in FIG. 1B.

In both methods, a crosslinker is used to crosslink monomers or dimers of streptavidin. The selection of crosslinker length is critical for achieving primarily intramolecular crosslinking within a tetramer of streptavidin (e.g., crosslinking two monomers of a streptavidin tetramer or crosslinking two dimers of a streptavidin tetramer). That is, the crosslinker must have a length sufficient to generate said intramolecular linkages without generating intermolecular linkages across tetramers, which can impact the binding of biotinylated affinity agents to the streptavidin binding sites.

A number of crosslinkers are suitable for use in the methods described herein, provided that said crosslinkers are a length that can achieve intramolecular crosslinking with minimal intermolecular crosslinking as described above. The bifunctional crosslinkers used herein may be a homo-functional crosslinker, i.e., the reactive groups are the same, or a hetero-functional crosslinker, i.e., the reactive groups are different. In some embodiments, the homo-functional crosslinkers contain amine-to-amine reactive groups or sulfhydryl-to-sulfhydryl reactive groups. In some embodiments, the hetero-functional crosslinkers contain amine-to-sulfhydryl reactive groups or carboxyl-to-amine reactive groups.

As would be understood by one of ordinary skill in the art, amine reactive groups of a crosslinker form a stable bond with primary amines, such as those found on lysine side chains or N-terminal amines. Sulfhydryl reactive groups of a crosslinker form stable bonds with exposed cysteine residues. Carboxyl reactive groups of a crosslinker form stable bonds with carboxyl-terminal ends of a protein or on aspartate or glutamate side chains. Accordingly, an amine-to-amine reactive crosslinker may be used to generate a linkage between two amine groups present in a streptavidin tetramer. Similarly, a sulfhydryl-to-sulfhydryl reactive crosslinker may be used to generate a linkage between two sulfhydryl groups present in a streptavidin tetramer. An amine-to-sulfhydryl reactive crosslinker may be used to generate a linkage between an amine group and a sulfhydryl group present in a streptavidin tetramer. A carboxyl-to-amine reactive crosslinker may be used to generate a linkage between a carboxyl group and an amine group present in a streptavidin tetramer.

The length of a crosslinker may be measured by the length of the spacer between the two reactive groups. In some embodiments, the crosslinker has a length that is between 1 Å to 30 Å. That is, in some embodiments, the crosslinker may be a homo-functional crosslinker with a spacer arm length of between 1-30 Å. In some embodiments, the crosslinker may be a hetero-functional crosslinker with a spacer arm length of between 1-30 Å.

For example, but not by way of limitation, the crosslinker may be selected from the group comprising: succinimydyl 3-(2-pyridyldithio)propionate, tris-(succinimidyl)aminotriacetate, tris(2-maleimidoethyl)amine, p-malemidiophenylisocyanate, N-hydroxysuccinimide, succinimidyl 4-(p-maleimidophenyl)butyrate, succinimidyl 6-((beta-maleimidopropionamideo)hexanoate), succinimidyl 4-(N-maleimidomethyl)cyclohexane-1-carboxy-(6-amidocaproate), succinimidyl iodoacetate, succinimidyl 3-(bromoacetamido) propionate, 1,11-bismaleimido-triethyleneglycol, 1,8-bismaleimido-diethyleneglycol, dithiobismaleimidoethane, 1,4-bismaleimidobutane, bismaleimidohexane, succinimidyl(4-iodoacetyl)aminobenzoate, sulfosuccinimidyl(4-iodoacetyl)aminobenzoate, N-gamma-maleimidobutyryl-oxysulfosuccinimide ester, bismaleimidoethane, sulfosuccinimidyl 4-(N-maleimidophenyl) butyrate, m-maleimidobenzoyl-N-hydroxysulfosuccinimide ester, m-maleimidobenzoyl-N-hydroxysuccinimide ester, N-gamma-maleimidobutyrl-oxysuccinimide ester, N-epsilon-malemidocaproyl-oxysuccinimide ester, N-epsilon-maleimidocaproyl-oxysulfosuccinimide ester, N-beta-maleimidopropyl-oxysuccinimide ester, N-alpha-maleimidoacetoxysuccinimide ester, 1,8-bismaleimido-diethyleneglycol, dithiobismaleimidoethane, 1,4-bismaleimidobutane, bismaleimidohexane, succinimidyl(4-iodoacetyl)aminobenzoate, sulfosuccinimidyl(4-iodoacetyl)aminobenzoate, N-gamma-maleimidobutyryl-oxysulfosuccinimide ester, bismaleimidoethane, sulfosuccinimidyl 4-(N-maleimidophenyl) butyrate, m-maleimidobenzoyl-N-hydroxysulfosuccinimide ester, m-maleimidobenzoyl-N-hydroxysuccinimide ester, N-gamma-maleimidobutyryl oxysuccinimide ester, N-epsilon-maleimidocaproyl-oxysuccinimide ester, N-epsilon-maleimidocaproyl-oxysulfosuccinimide ester, 4-(4-N-maleimidophenyl) butyric acid hydrazide, 3-(2-pyridyldithio) propionyl hydrazide, N-beta-maleimidopropyl-oxysuccinimide ester, N-beta, maleimidopropionic acid hydrazide, N-alpha-maleimidoacet-oxysuccinimide ester, N-kappa-maleimidoundecanoic acid hydrazide, N-epsilon-maleimidocaproic acid hydrazide, succinimidyl 3-(2-pyridyldithio) propionate, succinimidyl 6-(3 (2-pyridyldithio) propionamido) hexanoate, 6-(3′-(2-pyridyldithio) propionamido) hexanoate, bis(sulfosuccinimidyl) 2,2,7,7-suberate-d4, sulfosuccinimidyl bis(sulfosuccinimidyl) glutarate-d0, bis(sulfosuccinimidyl) suberate-d0,3,3′-dithiobis(sulfosuccinimidyl propionate), ethylene glycol bis(sulfosuccinimidylsuccinate), ethylene glycol bis(succinimidyl succinate), 4-succinimidyloxycarbonyl-alpha-methyl-alpha (2-pyridyldithio) toluene, N-kappa-maleimidoundecanoyl-oxysulfosuccinimide ester, dimethyl subermidate, disuccinimidyl tartrate, tert-butyl disuccinimidyl phenyl phosphonate, disuccinimidyl phenyl phosphonic acid, sulfosucciniidyl 6-(4′-azido-2′-nitrophenylamino) hexanoate, disuccinimidyl glutarate, 1-ethyl-3-(3-dimethylaminopropyl) carbodiimide hydrochloride, 1-ethyl-3-(3-dimethylaminopropyl) carbodiimide hydrochloride, N-hydroxysulfosuccinimide, and ethylene glycol diglycidyl ether.

In some embodiments, the crosslinker is a poly(ethylene glycol) diglycidyl ether polymer including n ethylene oxide units. In some embodiments, n is from 1 to 50 ethylene oxide units (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ethylene glycol units). In some embodiments, a poly(ethylene glycol) diglycidyl polymer may be referred to by the average number of ethylene oxide units. For example, a poly(ethylene glycol) diglycidyl polymer composition with an average of 9 ethylene oxide units may be referred to as n=9 poly(ethylene glycol) diglycidyl.

Crosslinked streptavidin molecules may be conjugated to the surface of any chromatographic material, such as a particle, monolith, or membrane.

In preferred embodiments, the chromatographic material comprises nonporous particles. In some embodiments, the nonporous particles comprise a polymer core. In some embodiments, the nonporous polymer core has a gradient composition. In some embodiments, the nonporous polymer core comprises divinylbenzene (80%) and polystyrene. Alternatively, the nonporous particles may comprise an inorganic core, such as silica. In some embodiments, the nonporous particles may comprise a hybrid organic/inorganic core. In some embodiments, the nonporous particles have an average diameter of between 1-10 μm.

The crosslinked streptavidin molecules may be conjugated to a hydrophilic surface, such as a hydrophilic surface present on a particle, monolith, or membrane, using methods known in the art. In some embodiments, the crosslinked streptavidin molecules may be conjugated to a hydrophilic surface with an epoxy linker, for example, an epoxy linker of Formula I:

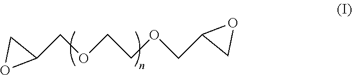

wherein n is between 1-12. In some embodiments, n is 1, 4, or 9. In some embodiments, n is 1.

In some embodiments, the chromatographic material comprises porous particles. Porous particles may have a pore volume of at least 0.1 cc/g (e.g., at least 0.2 cc/g, at least 0.3 cc/g, at least 0.4 cc/g, at least 0.5 cc/g, at least 0.6 cc/g, at least 0.7 cc/g, at least 0.7 cc/g, at least 0.8 cc/g, at least 0.9 cc/g, at least 1.0 cc/g, at least 1.1 cc/g, at least 1.2 cc/g, at least 1.3 cc/g, at least 1.4 cc/g, at least 1.5 cc/g, at least 1.6 cc/g, at least 1.7 cc/g, at least 1.8 cc/g, at least 1.9 cc/g, at least 2.0 cc/g, etc.). In some embodiments, porous particles may have a pore volume of at least 1.3 cc/g. The porous particles may comprise a polymer core, a silica core, or a hybrid organic/inorganic core. In some embodiments, the porous particles have an average diameter of between 1-10 μm.

In any of the above embodiments, the crosslinked streptavidin molecules may be present at a concentration of between 1 to 8 μg/mg of particle. In some embodiments, the crosslinked streptavidin molecules may be present at a concentration of between 2 to 6 μg/mg of particle.

In some embodiments, the chromatographic material comprises a monolith. In said embodiments, the crosslinked streptavidin molecules may be conjugated to a surface present in the monolith.

In some embodiments, the chromatographic material comprises a membrane. In said embodiments, the crosslinked streptavidin molecules may be conjugated to a surface of the membrane.

As described above, crosslinking of the streptavidin molecules may be performed prior to conjugation to the particle, monolith, or membrane or after conjugation to the particle, monolith, or membrane. Streptavidin molecules or crosslinked streptavidin molecules may be conjugated to the surface of the particle, monolith, or membrane using methods known in the art. For example, but not by way of limitation, the crosslinked streptavidin molecules or streptavidin molecules may be conjugated to the particle, monolith, or membrane using an epoxide reaction.

In some preferred embodiments, the crosslinked streptavidin molecules are conjugated to the hydrophilic surface of a nonporous polymer particle. Said nonporous polymer particles and the synthesis thereof are further described in PCT Publication No. WO 2024/224364, incorporated herein by reference. Example 1 further describes methods of preparing nonporous polymer particles that may be used in conjunction with the present technology.

The resultant particles, monoliths, or membranes comprising crosslinked streptavidin molecules provide a plurality of accessible streptavidin binding sites. Said streptavidin binding sites may be contacted with biotin or a biotinylated molecule, such as a biotinylated affinity agent. Due to the crosslinked streptavidin, the particles, monoliths, or membranes described herein result in reduced or no leachate of streptavidin monomers from the particle, monolith, or membrane.

### Methods of Determining Streptavidin Leachate

Streptavidin leachate can be monitored by UV absorbance, for example at 280 nm. For example, particles, monoliths, or membranes comprising crosslinked streptavidin may be washed with a mobile phase and the eluent monitored by UV at 280 nm. As the crosslinked streptavidin of the particles, monoliths, or membranes described herein reduce streptavidin leachate, little to no absorbance following washing with the mobile phase will be detected.

Streptavidin leachate can be observed and measured by flowing a mobile phase across the column comprising a plurality of crosslinked streptavidin-conjugated particles, a crosslinked streptavidin-conjugated monolith, or a crosslinked streptavidin-conjugated membrane. Eluent from the column, monolith, or membrane may be monitored by UV absorbance at 280 nm for the presence of streptavidin monomers in the eluent. Eluent may further be detected using a mass spectrometer to detect the presence of streptavidin monomers as determined by molecular weight.

The mobile phase may comprise a buffer, such as phosphate buffered saline (PBS). The mobile phase may further comprise an organic solvent such as, but not limited to, acetonitrile, methanol, or isopropanol. The mobile phase may further comprise an acid, such as phosphoric acid. Additionally or alternatively, the mobile phase may comprise a detergent. The concentration of the organic solvent, detergent, and/or acid may be adjusted as would be understood by one of ordinary skill in the art.

The crosslinked streptavidin-conjugated particles, monoliths, or membranes described herein may result in no detectable streptavidin leachate as determined by mass spectrometry.

The crosslinked streptavidin-conjugated particles, monoliths, or membranes described herein may result in reduced streptavidin leachate as measured by UV absorbance. In some embodiments, the UV absorbance following washing with a mobile phase is less than 10 mAU.

Alternatively, reduction in streptavidin leachate may be measured as a reduction in peak area following washing with a mobile phase. For said measurement, a comparison is made between a particle, membrane, or monolith having crosslinked streptavidin versus a particle, membrane, or monolith having streptavidin that is not crosslinked. In some embodiments, the 4^{th} peak is used as the measurement for determining reduction in streptavidin leachate. In some embodiments, the crosslinked streptavidin-conjugated particle, membrane, or monolith results in an 85% reduction in absorbance as measured at the 4^{th} peak. In some embodiments, the crosslinked streptavidin-conjugated particle, membrane, or monolith results in a 90% or 95% reduction in absorbance as measured at the 4^{th} peak.

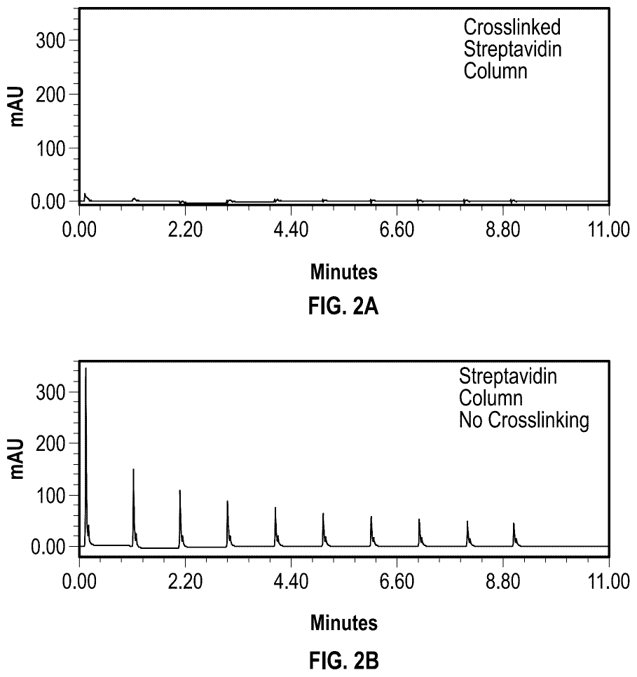

Examples 2 and 3 further describes methods of determining streptavidin leachate from a column comprising a plurality of crosslinked streptavidin-conjugated particles as compared to a column comprising a plurality of streptavidin-conjugated particles. FIG. 2 demonstrates the reduction in streptavidin leachate observed with the crosslinked streptavidin-conjugated particles.

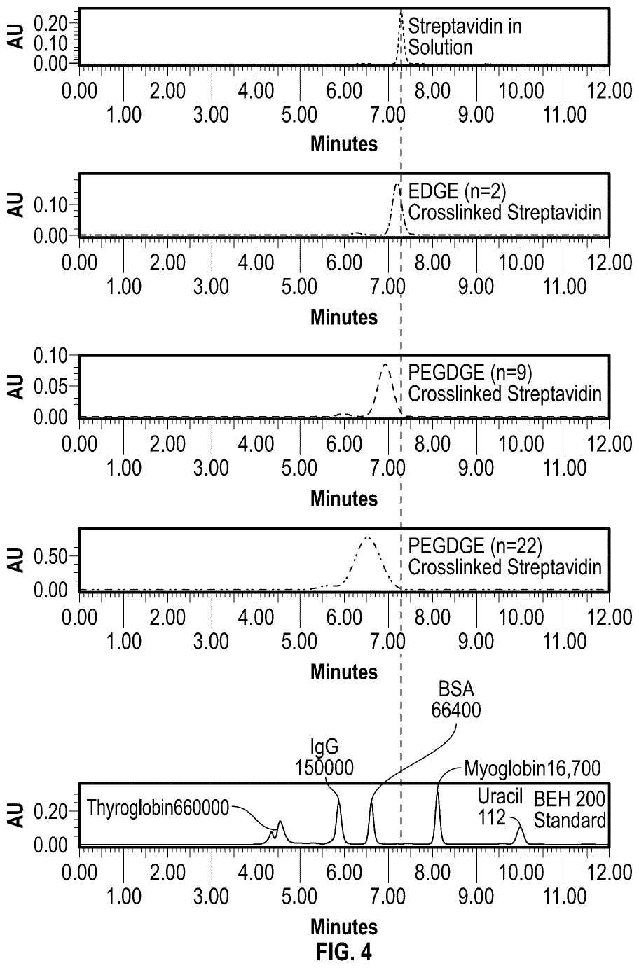

Example 4 demonstrates the impact of crosslinker length on polydispersity of crosslinked streptavidin molecules. FIG. 4 shows that ethylene glycol diglycidyl ether (EGDGE) crosslinkers minimize peak broadening effects in SEC experiments, and maintain similar elution times to free streptavidin.

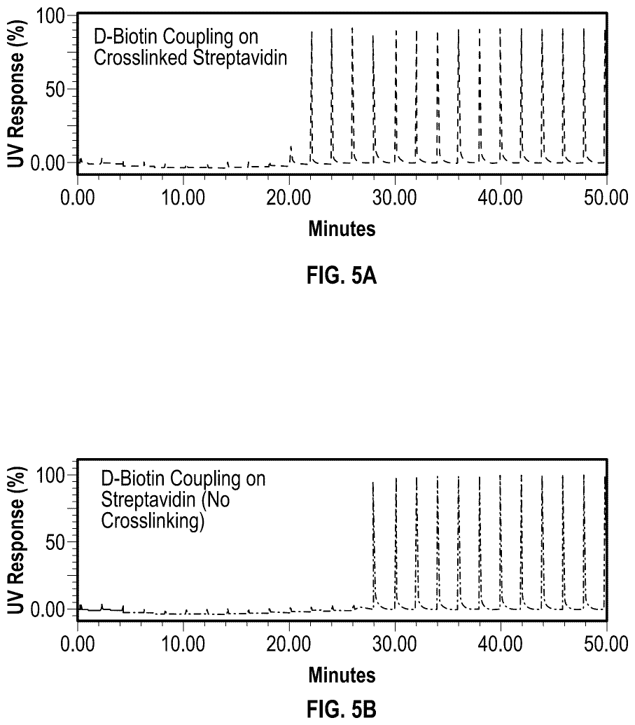

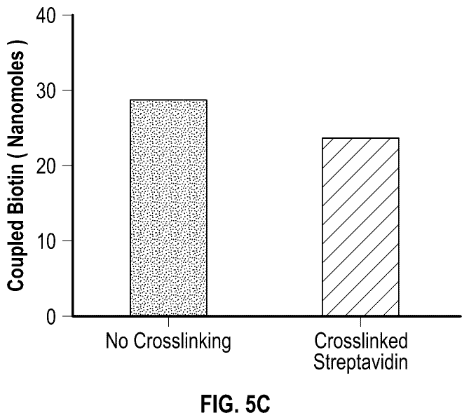

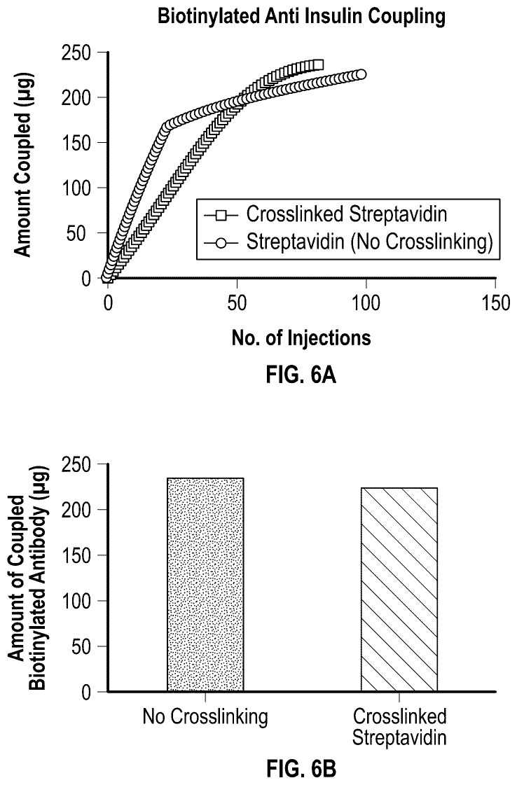

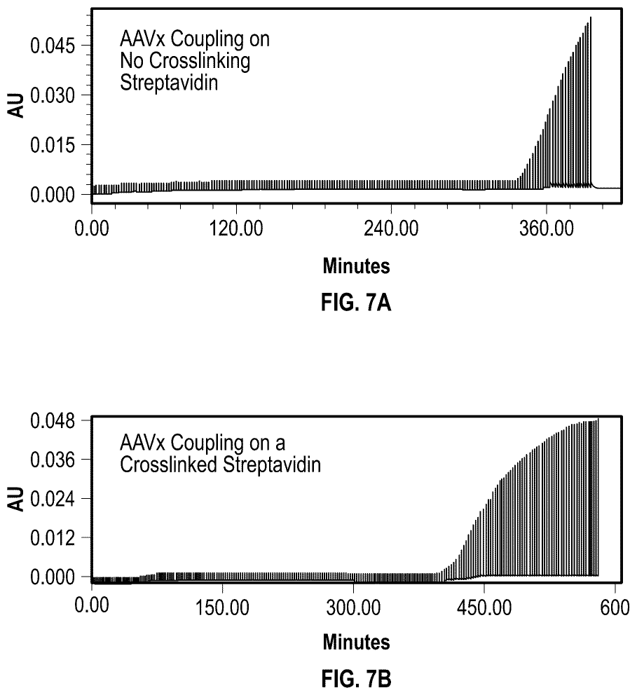

Example 5 demonstrates the ability for biotin (FIG. 5A-5C), a biotinylated antibody (FIG. 6A-6B), or a biotinylated nanobody (FIG. 7A-7B) to bind to crosslinked streptavidin as compared to streptavidin (not crosslinked). Example 5 further demonstrates crosslinking streptavidin does not reduce its binding capacity for biotin or biotinylated antibodies.

### Systems of Crosslinked Streptavidin-Conjugated Particles, Monoliths, or Membranes

The crosslinked streptavidin-conjugated particles, monoliths, or membranes may be used to prepare an affinity chromatographic column, an affinity monolith, or an affinity membrane, respectively.

In one aspect, provided herein are chromatographic columns comprising a plurality of crosslinked streptavidin-conjugated chromatographic materials. Said chromatographic materials may be porous or nonporous particles.

In one aspect, provided herein is a chromatographic column comprising a plurality of crosslinked streptavidin-conjugated particles disclosed herein. Said chromatographic column may further be contacted with one or more biotinylated affinity agents to generate an affinity chromatographic column. For example, but not by way of limitation, a chromatographic column comprising a plurality of crosslinked streptavidin-conjugated particles may be contacted with a solution comprising a biotinylated affinity agent. Said biotinylated affinity agent will bind to accessible binding sites of the crosslinked streptavidin molecules, resulting in an affinity chromatographic column. Throughout use of the affinity chromatographic column, said affinity chromatographic column has reduced or no detectable streptavidin leachate as measured by UV absorbance and/or mass spectrometry.

In one aspect, provided herein a crosslinked streptavidin-conjugated monolith. Said monolith may further be contacted with one or more biotinylated affinity agents to generate an affinity monolith. Said biotinylated affinity agents will bind to accessible binding sites of the crosslinked streptavidin molecules present in the monolith, thereby generating an affinity monolith. Throughout use of the affinity monolith, said affinity monolith has reduced or no detectable streptavidin leachate as measured by UV absorbance and/or mass spectrometry.

In one aspect, provided herein is a crosslinked streptavidin-conjugated membrane. Said membrane may further be contacted with one or more biotinylated affinity agents to generate an affinity membrane. Said biotinylated affinity agents will bind to accessible binding sites of the crosslinked streptavidin molecules present in the membrane, thereby generating an affinity membrane. Throughout use of the affinity membrane, said affinity membrane has reduced or no detectable streptavidin leachate as measured by UV absorbance and/or mass spectrometry.

As used herein, the term “biotinylated affinity agent” refers to a biotinylated molecule that can specifically bind to a target antigen or complementary nucleic acid sequence. The biotinylated affinity agent may be a biotinylated antibody or antigen-binding fragment thereof or a biotinylated oligonucleotide. The preparation of biotinylated molecules is a process well known and understood in the art. In some embodiments, the molecule is biotinylated with a biotin derivative, including but not limited to iminobiotin, desthiobiotin, disulfide biotin azide, disulfide biotin alkyne or other biotin derivatives.

In some embodiments, the biotinylated affinity agent is a biotinylated antibody, or a biotinylated antigen-binding fragment thereof. In some embodiments, the biotinylated affinity agent is a biotinylated oligonucleotide.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, nanobodies, humanized antibodies, heteroconjugate antibodies (e.g., bi-, tri-, and quad-specific antibodies, diabodies), and antigen-binding fragments of antibodies, including, for example, Fab′, F(ab′)2, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)2 fragments refer to antibody fragments that lack the Fc portion of an intact antibody.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)2, scFv, a camelid, an affibody, a nanobody, an aptamer, or a domain antibody.

As used herein, the term “bispecific antibody” refers to an antibody that is capable of binding at least two different antigens.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

For the chromatographic columns, a number of column sizes and materials are suitable for use. In some embodiments, the column material is stainless steel, polyetheretherketone (PEEK) lined steel, titanium, or a stainless steel alloy such as MP35n. In some embodiments, the column material is plastic. In some embodiments, the column has an internal diameter ranging from 75 μm to 4.6 mm. In some embodiments, the column has a length between 5 to 300 mm. The column surface can be unmodified or modified to generate a high-performance surface. Chromatography columns suitable for use with the methods disclosed herein are compatible with any standard liquid chromatography system, including high-performance liquid chromatography (HPLC) systems, ultra-high performance liquid chromatography (UHPLC) systems, and fast protein liquid chromatography (FPLC) systems.

In some embodiments, the liquid chromatography system is connected in series to a detector. Detectors suitable for use in the methods disclosed herein include detectors for ultraviolet spectroscopy, fluorescence spectroscopy, and/or mass spectrometry. In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected to one or more of the detectors in series.

In some embodiments, the interior surfaces of the column are treated to reduce non-specific binding and enhance overall efficiency of the liquid chromatography system. In particular, an alkylsilyl coating or other high performance surface is provided to limit or reduce non-specific binding of a sample with walls or interior surfaces of a column body. Without wishing to be bound by theory, it is believed that an alkylsilyl coating covering metal surfaces prevent or minimize contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors.

In one embodiment, the alkylsilyl coating comprises a hydrophilic, non-ionic layer of polyethylene glycol silane. In another embodiment, the alkylsilyl coating is formed from one or more of the following precursor materials bis(trichlorosilyl) ethane or bis(trimethoxysilyl) ethane. Other embodiments of alkylsilyl coatings suitable for use with the present technology are described in U.S. Patent Publication No. 2019/0086371 and U.S. Application Publication No. 2022/0118443.

The affinity chromatographic columns, affinity monoliths, or affinity membranes described herein may be used for affinity capture of a target analyte, i.e., an analyte that binds to the biotinylated affinity agent. For example, but not by way of limitation, an affinity chromatographic column, affinity monolith, or affinity membrane may be contacted with a solution comprising a target analyte, such that the target analyte binds to the biotinylated affinity agent. The column, monolith, or membrane may be washed with a suitable washing buffer, such that additional compounds present in the solution are washed out of the column, monolith, or membrane. The target analyte may be eluted from the column, monolith, or membrane by washing said column, monolith, or membrane with an elution buffer. In some embodiments, the target analyte is eluted such that there is no streptavidin leachate, i.e., streptavidin monomers, present in the eluate. Streptavidin leachate may be detected as described herein, including via monitoring with UV absorbance and/or mass spectrometry.

### Examples

The following examples are meant to illustrate the invention and are not meant to limit the invention in any way.

### Example 1: Addition of an Epoxy Linker to Hydrophilic Particles

The nonporous, epoxy-modified hydrophilic particles for use in the disclosed methods were prepared as follows. As a first step, 1500 g of reagent alcohol (90% ethanol, ~5% methanol, and ~5% isopropanol), 45.1 g of polyvinylpyrrolidone (PVP-40), 4.8 g of 2,2′-Azobis(2-methylpropionitrile), 5.9 g of surfactant (Triton™N-57, available from Dow, Inc), and 81.7 g of styrene were charged into a reactor. After the reactor was purged with nitrogen gas, the reaction mixture was heated to and maintained at 70° C. with stirring for 3 hours.

After three hours, a solution containing 110.4 g of divinylbenzene 80 (DVB), 39.7 g of PVP-40, 510 g of reagent alcohol, and 100.2 g of p-xylene was added to the reaction mixture at a constant flow rate over two hours. Following this step, a primer coating solution containing 26.0 g of glycidyl methacrylate (GMA), 26.0 g of ethylene glycol dimethacrylate (EDMA), 36.4 g of PVP-40, and 560 g of reagent alcohol were added to the reaction mixture at a constant flow rate over 1.5 hours.

The reaction mixture was maintained at 70° C. for a total of 20 hours, after which the particles were separated from the reaction slurry by filtration. The particles were then washed sequentially with methanol, tetrahydrofuran (THF), and acetone. The final product was dried in a vacuum oven at 45° C., resulting in monodisperse 3.5 μm polymer particles. These particles contain a gradient polystyrene/DVB core with a poly(GMA/EDMA) primer (FIG. 1). While the above reaction conditions generate 3.5 μm polymer particles, it is understood that particles ranging in sizes from 1.5 μm to 8 μm are within the scope of the disclosure. By altering the concentrations of PVP-40, 2′2-Azobis(2-methylpropionitrile), and Triton N-57, one of ordinary skill in the art could generate a range of particle sizes.

The resultant 3.5 μm, polystyrene/DVB particles with the poly(GMA/EDMA) primer were then coated with a hydrophilic layer. 70 g of the particles were hydrolyzed in 0.5M H2SO4 at 60° C. for 1-20 hours. The hydrolyzed particles were washed sequentially with MilliQ water and methanol, and then dried under vacuum at 45° C. overnight. The dried particles were added into a 1 L three-necked round bottom flask with an overhead stirring motor, stirring shaft, and stir blade, a water-cooled condenser, a nitrogen inlet, and a probe-controlled heating mantle. 700 mL of anhydrous diglyme (diethylene glycol dimethyl ether) was added, the flask sealed and purged with nitrogen for 15 minutes with moderate stirring. 2.0 g of potassium tert-butoxide was added, and the reaction was raised to 70° C. To generate the hydrophilic layer, a mixture of 10.5 g glycidol, 2.6 g of glyceroltriglycidyl ether, and 14.9 g of anhydrous diglyme was prepared separately and added to the particle mixture in four equal aliquots in 30-minute intervals. The reaction was held at 70° C. for 20 hours, cooled to RT, and filtered. The resulting particles were washed sequentially with water 6 times, methanol 3 times, and then dried under vacuum overnight at 45° C. The following procedure results in a hydrophilic layer that is 2-4% (by weight) of the entire particle.

20 g of the resultant 3.5 μm particles with the hydrophilic coating were added to a mixture of 100 g of ethylene glycol diglycidyl ether (EGDGE) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable functionalization of the particle surface.

### Example 2: Preparation of Crosslinked Streptavidin-Conjugated Particles (Method 1)

Crosslinked streptavidin was prepared using an ethylene glycol diglycidyl ether (EGDGE) crosslinker. To a 10 mg/mL streptavidin solution in sodium carbonate-bicarbonate buffer (pH 9.4) was added ethylene glycol diglycidyl ether (340:1 to 1700:1 molar ratio with streptavidin). The solution was heated at 37° C. with gentle mixing for 4-14 hours. Following the incubation, 83 mg of ethanolamine in 311 μL of sodium carbonate-bicarbonate buffer was added and the reaction was stirred at RT for 1 hour. Then, the buffer was exchanged with 100 mM PBS (pH 7.2) by consecutive wash/centrifugation cycles.

The particles as prepared in Example 1 were then conjugated with the crosslinked streptavidin molecules. 2 g of particles were dispersed in 9.5 mL of a 100 mM sodium carbonate-bicarbonate buffer (pH 9.4). To this, 2 mL of a 10 mg/mL solution of crosslinked streptavidin was added. Next, 28.5 mL of sodium carbonate-bicarbonate buffer containing sodium sulfate, a salting out agent, was added dropwise. The reaction was stirred for 20 hours at 37° C.

Following the 20 h reaction, 1.4 g of ethanolamine in 4.9 mL of sodium carbonate-bicarbonate buffer was added and the reaction was stirred at RT for 3 hours. Particles were isolated by filtration and washed. The washing process comprises 4 steps. Step 1 is a 3× wash with water (pH 4) adjusted with HCl. Step 2 is a 3× wash with water/acetonitrile (4:1) with 1% phosphoric acid. Step 3 is a 3× wash with water. And Step 4 is a 2× wash with storage buffer (100 mM PBS, pH 7.2, 0.02% sodium azide). The particles were then stored in a sealed container as a slurry in storage buffer (~10 mL buffer/g of particle) at 4° C. Streptavidin coverage of the particles was determined using a standard bicinchoninic acid (BCA) assay. Streptavidin leachate was assessed by monitoring the eluent from the column following 10, 25 μL injections of 20% acetonitrile and 1% phosphoric acid. Table 1 shows the effects of the crosslinker molar ratio and the reaction time on the streptavidin coverage on a given particle, as well as the resultant streptavidin leachate from the column.

TABLE 1
Crosslinked streptavidin particles prepared
with crosslinked streptavidin

| | EGDGE/ | Reaction | Streptavidin | % Decrease in |
|---|---|---|---|---|
| | Streptavidin | Time | Coverage | 4^{th} Peak |
| Example # | Molar Ratio | (h) | (μg/mg particle) | Area* |
| 2a | 340 | 4 | 5.7 | 86 |
| 2b | 1700 | 4 | 13.9 | 62 |
| 2c | 340 | 14 | 8.6 | 80 |
| 2d | 1700 | 14 | 13.4 | 54 |

*Compared to particles prepared without streptavidin crosslinking.

FIG. 2 demonstrates the reduction in leachate observed with crosslinked streptavidin-conjugated particles (top) as compared to streptavidin-conjugated particles (bottom). As measured at the 4^{th} peak, the absorbance for the crosslinked streptavidin-conjugated particles is 3.5 mAU as compared to 91 mAU for the streptavidin-conjugated particles.

The eluent of a column having crosslinked streptavidin-conjugated particles and the eluent of a column having streptavidin-conjugated particles were further analyzed with mass spectrometry for the presence of streptavidin monomers. The respective columns were first washed with 0.1% formic acid (FA) at 0.2 mL/min followed by a 10 μL injection of 50% MeCN with 0.1% (FA). Next, a 3 minute gradient from aqueous 0.1% FA to 80% MeCN in 0.1% FA at a flow rate of 0.2 mL/min was applied. Next, a 15 minute gradient from aqueous 0.1% FA to 80% MeCN in 0.1% FA at a flow rate of 0.2 mL/min was applied. Eluent was monitored with UV followed by mass spectrometry.

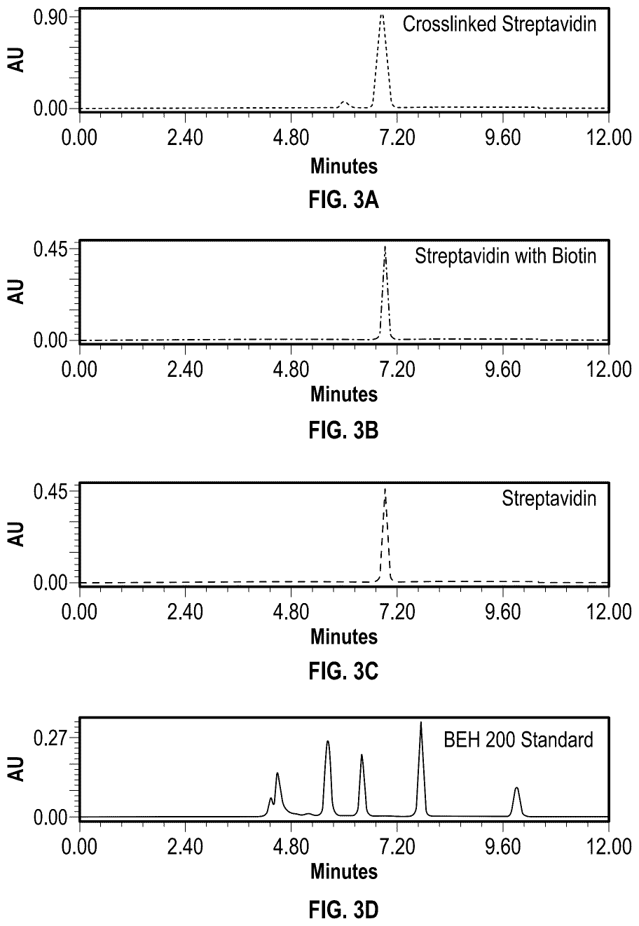

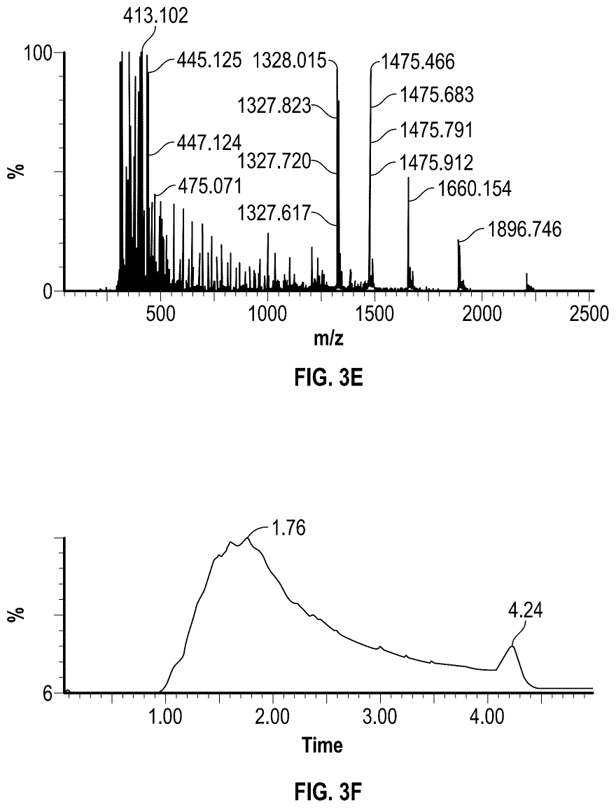

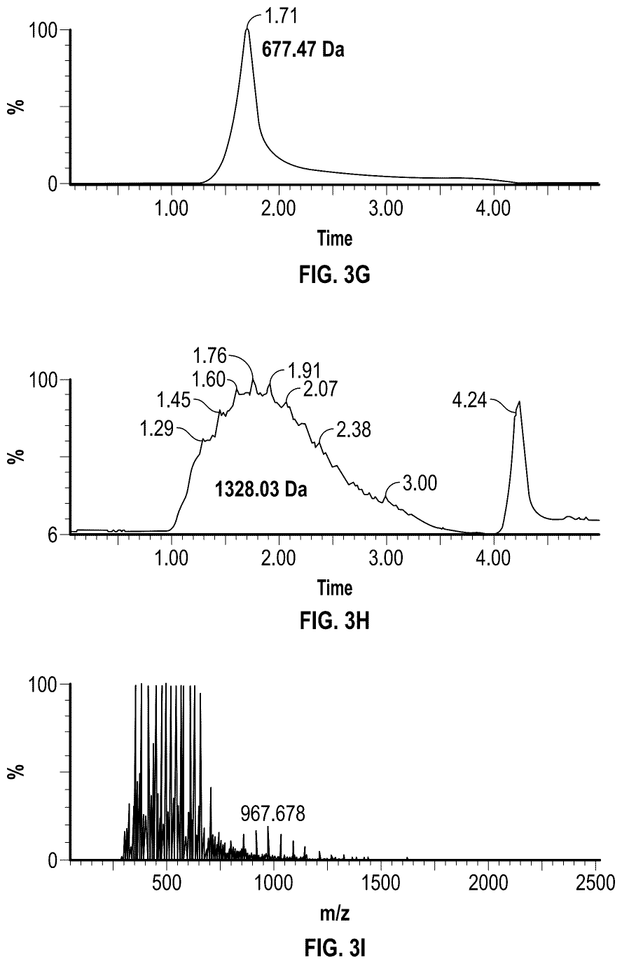

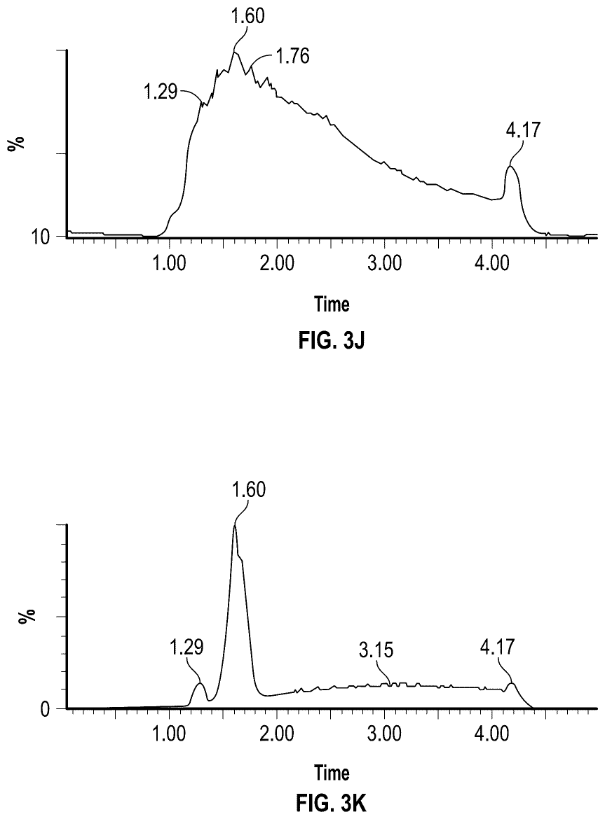

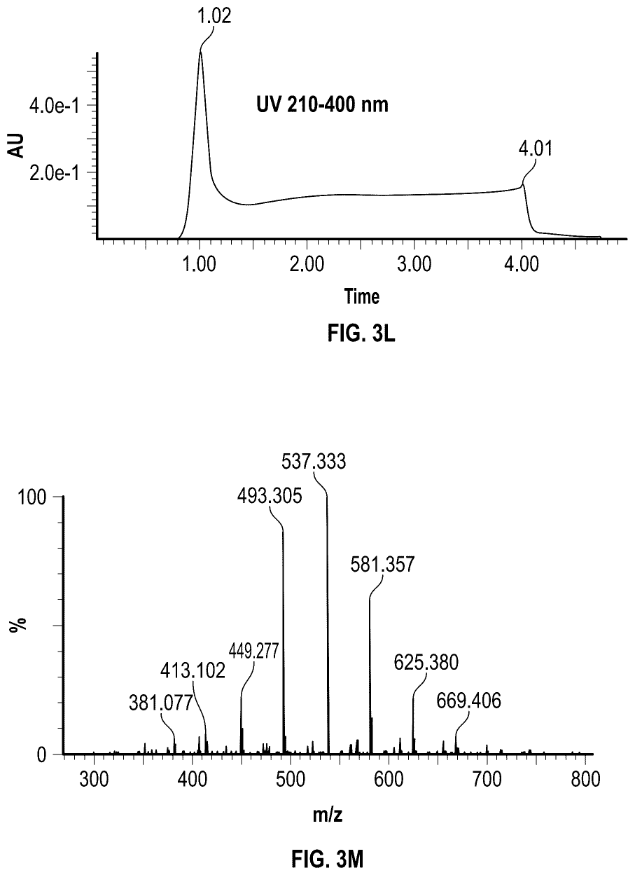

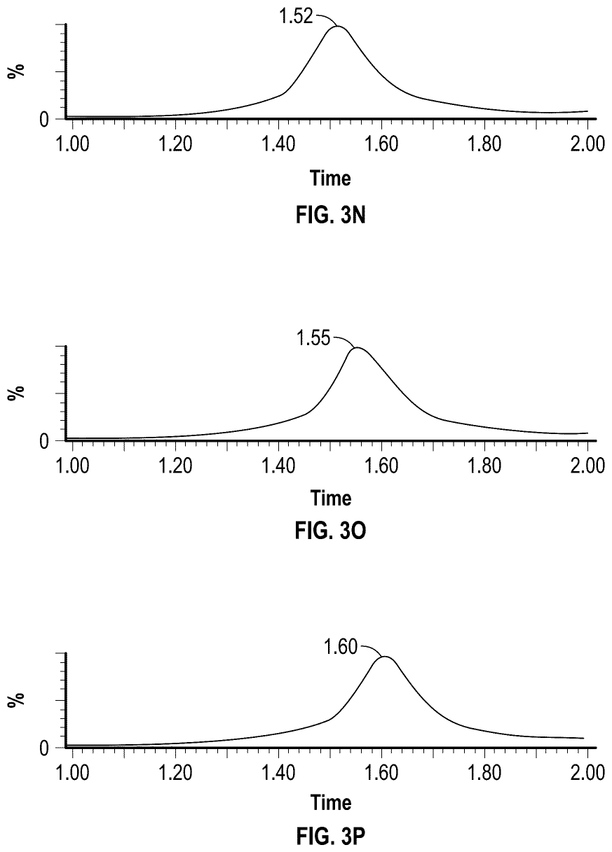

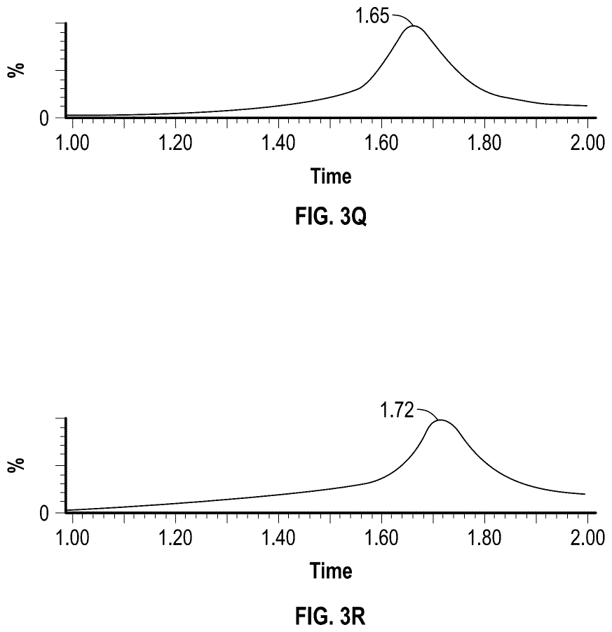

FIGS. 3A-3D demonstrate that the solution of streptavidin molecules treated with a crosslinker had a slightly larger molecular size as compared to streptavidin alone or streptavidin bound with biotin as measured by SEC. FIG. 3E shows the mass spectrometry spectra of the eluent from a column of particles coated with non-crosslinked streptavidin. Signals corresponding to both the hydrophilic particle and the streptavidin monomer are observed. FIGS. 3F-3H track the elution of species of specific masses from the column. FIG. 3I shows the mass spectrometry spectra of the eluent from a column of particles coated with crosslinked streptavidin. Only signals corresponding to the hydrophilic particles were present (i.e. no signals correspond to streptavidin). FIGS. 3J-3L show the TIC MS, BPI MS, and the UV-Vis spectra of the eluent. FIGS. 3M-3R track the elution of species of specific masses from the column. Each tracked mass elutes at a different time.

### Example 3: Preparation of Crosslinked Streptavidin-Conjugated Particles (Method 2)

Particles prepared as described in Example 1 were functionalized with streptavidin (as received; not crosslinked) followed by crosslinking with an ethylene glycol diglycidyl ether crosslinker. 2 g of particles were dispersed in 9.5 mL of a 100 mM sodium carbonate-bicarbonate buffer (pH 9.4). To this, 2 mL of a 10 mg/mL solution of streptavidin was added. Next, 28.5 mL of sodium carbonate-bicarbonate buffer containing sodium sulfate, a salting out agent, was added dropwise. The reaction was then stirred for 20 hours at 37° C.

Following the 20 h reaction, 1.4 g of ethanolamine in 4.9 mL of sodium carbonate-bicarbonate buffer was added and the reaction was stirred at RT for 3 hours. Particles were then isolated by filtration and washed. The washing process comprises 4 steps. Step 1 is a 3× wash with water (pH 4) adjusted with HCl. Step 2 is a 3× wash with water/acetonitrile (4:1) with 1% phosphoric acid. Step 3 is a 3× wash with water. And Step 4 is a 2× wash with storage buffer (100 mM PBS, pH 7.2, 0.02% sodium azide). The particles were then stored in a sealed container as a slurry in storage buffer (~10 mL buffer/g of particle) at 4° C. Streptavidin coverage of the particles was determined using a standard bicinchoninic acid (BCA) assay.

Streptavidin on the particles was then crosslinked with ethylene glycol diglycidyl ether as follows. 1.2 g of the streptavidin-conjugated particles were dispersed in 100 mM sodium carbonate-bicarbonate buffer, pH 9.4 (~10 mL buffer/g of particle). Ethylene glycol diglycidyl ether was added at a final concentration of 30-450 mM. The reaction was then stirred at 37° C. for 4-16 hours. Following the reaction, 0.4 g ethanolamine in 1.5 mL sodium carbonate-bicarbonate buffer was added and the reaction was stirred at RT for 1 hour. Particles were then isolated by filtration and washed sequentially 4 times with water and 2 times with storage buffer (100 mM PBS, pH 7.2, 0.02% sodium azide). The particles were stored in a sealed container as a slurry in storage buffer (~10 mL buffer/g of particle) at 4° C. The effects of the crosslinker concentration and the reaction time on streptavidin leachate from the column is shown in Table 2. Streptavidin leachate was assessed by monitoring the eluent from the column following 10, 25 μL injections of 20% acetonitrile and 1% phosphoric acid.

TABLE 2
Crosslinked streptavidin particles prepared with crosslinking
after streptavidin functionalization.

| | | | Streptavidin | |
|---|---|---|---|---|
| | EGDGE | Reaction | Coverage | % Decrease in |
| | Concentration | Time | (μg/mg | 4^{th} Peak |
| Example # | (mM) | (h) | particle) | Area* |
| 3a | 30 | 4 | 6.4 | 38 |
| 3b | 30 | 14 | 6.4 | 41 |
| 3c | 150 | 4 | 6.4 | 50 |
| 3d | 150 | 14 | 6.4 | 44 |
| 3e | 450 | 4 | 6.4 | 53 |

*Compared to the particles prepared without streptavidin crosslinking.

### Example 4: Effect of Crosslinker Length on Streptavidin Properties

To investigate the impact of different crosslinkers on the uniformity properties of the streptavidin, crosslinked streptavidin was prepared crosslinked using ethylene glycol diglycidyl ether polymers including: (1) one ethylene oxide unit (n=1); (2) nine ethylene oxide units (n=9); and (3) twenty-two ethylene oxide units (n=22) were compared to crosslinked streptavidin and a BEH 200 standard. (FIG. 4).

As the length of the crosslinker increased, the streptavidin absorbance broadened, and shifted towards faster elution times, indicating the streptavidin varied more in size and was typically larger. In order to prevent streptavidin polydispersity from impacting experiment results, EGDGE (ethylene glycol diglycidyl ether) was utilized for crosslinking experiments in the following experiments.

### Example 5: Coupling of Biotin or a Biotinylated Antibody to Crosslinked Streptavidin

The ability for biotin or a biotinylated antibody to bind to accessible binding sites of crosslinked streptavidin was determined. Columns comprising either crosslinked streptavidin-conjugated particles or streptavidin-conjugated particles were tested for the ability to bind to biotin or a biotinylated antibody.

### Biotin

Columns were washed with a mobile phase of 100 mM sodium phosphate (pH 7.4) at a flow rate of 0.15 mL/min. D-biotin, at a concentration of 1 nmol/μL, was injected with 1 μL injections until saturation was observed as measured by UV at 210 nm. As shown in FIG. 5A-5B, D-biotin was able to bind to accessible binding sites of streptavidin for both columns. There was an approximate ~17% reduction in biotin binding to crosslinked streptavidin. After accounting for differences in streptavidin coverage of the particles, columns including crosslinked streptavidin particles displayed an approximately 7.3% reduction in biotin binding (FIG. 5B).

### Biotinylated Antibody

Columns were washed with a mobile phase of 100 mM sodium phosphate (pH 7.4) at a flow rate of 0.1 mL/min. A solution of biotinylated anti-insulin antibody at 1 μg/μL concentration was injected onto the column with 10 μL injections until saturation was observed as measured by UV at 280 nm. As shown in FIG. 6A, the biotinylated antibody was able to bind to accessible binding sites of streptavidin for both columns. There was an approximate 4% reduction in anti-insulin antibody binding after accounting for differences in streptavidin coverage between the particles (FIG. 6B).

### Biotinylated Nanobody

Columns were washed with a mobile phase of 100 mM sodium phosphate (pH 7.4) at a flow rate of 0.1 mL/min. A solution of biotinylated anti-AAVx nanobody at 1 μg/μL concentration was injected onto the column with 10 μL injections until saturation was observed as measured by UV at 280 nm. As shown in FIG. 7A-7B, the biotinylated anti-AAVx nanobody was able to bind to accessible binding sites of streptavidin for both columns. There was an approximate ~14% increase in anti-AAVx nanobody binding to the crosslinked streptavidin column after accounting for differences in streptavidin coverage between the particles.

The results of these experiments, viewed collectively, indicate that crosslinking streptavidin does not have a significant impact on biotin binding. Free biotin and biotinylated antibodies display a minor reduction in binding capacity compared to a column with non-crosslinked streptavidin. Moreover, as indicated by the experiments using biotinylated nanobodies, for certain affinity agents, crosslinking may increase biotin binding capacity.

### OTHER EMBODIMENTS

Various modifications and variations of the described invention will be apparent to those skilled in the art without departing from the scope of the invention. Although the invention has been described in connection with specific embodiments, it should not be unduly limited to such specific embodiments. Indeed, various modifications of the described modes for carrying out the invention that are obvious to those skilled in the art are intended to be within the scope of the invention.

Other embodiments are in the claims.

1. A method of generating crosslinked streptavidin particles, the method comprising:
a) contacting a plurality of streptavidin molecules with a crosslinker to generate a plurality of crosslinked streptavidin molecules; and
b) contacting the plurality of crosslinked streptavidin molecules with a plurality of nonporous particles to generate a plurality of crosslinked streptavidin-conjugated nonporous particles.

2. (canceled)

3. The method of claim 1, wherein the crosslinker is a homo-functional or a hetero-functional crosslinker.

4. The method of claim 3, wherein the crosslinker is a homo-functional crosslinker.

5. The method of claim 4, wherein the crosslinker is ethylene glycol diglycidyl ether.

6. The method of claim 1, wherein each particle of the plurality of particles comprises:
a nonporous polymer core;
a hydrophilic surface on an outer layer of the nonporous polymer core; and
wherein the particle has an average particle size between 1.0 μm to 10 μm.

7. The method of claim 6, wherein the nonporous polymer core has a gradient composition.

8. The method of claim 6, wherein the nonporous polymer core comprises divinylbenzene (80%) and polystyrene.

9. The method of claim 6, wherein the hydrophilic surface is selected from the group consisting of: (3-glycidyloxypropyl)trimethoxysilane, (3-glycidyloxypropyl)triethoxysilane, polyacrylate, glycidol, glyceroltriglycidyl ether, and poly(methyl acrylate).

10. The method of claim 1, wherein the plurality of crosslinked streptavidin molecules and/or plurality of streptavidin molecules are conjugated to the hydrophilic surface of the particle via an epoxy linker.

11. The method of claim 10, wherein the epoxy linker has a formula of 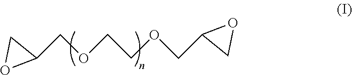
wherein n is between 1-12.

12. The method of claim 11, wherein n is 1, 4, or 9.

13. The method of claim 12, wherein n is 1.

14. The method of claim 1, wherein the plurality of crosslinked streptavidin molecules and/or plurality of streptavidin molecules conjugated to the hydrophilic surface provides a surface coverage of 2-6 μg/mg particle.

15. A chromatographic column comprising a column body formed of a metal or a metal alloy, the column body housing a plurality of crosslinked streptavidin-conjugated nonporous particles generated by the method of claim 1.

16. The chromatographic column of claim 15, wherein at least a portion of an interior surface of the column body is coated with an alkylsilyl material.

17. The chromatographic column of claim 16, further comprising frits within the column body, wherein the frits are coated with the alkylsilyl material.

18. The chromatographic column of claim 16, wherein the alkylsilyl material is a hydrophilic, non-ionic layer of polyethylene glycol silane.

19. The chromatographic column of claim 15, wherein the column has no detectable leachate of streptavidin as determined by UV absorbance.

20. The chromatographic column of claim 19, wherein the column has a leachate absorbance value of <10 mAU as measured by UV absorbance at 280 nm.

21. The chromatographic column of claim 1, wherein the column has at least an 85% reduction in leachate absorbance as compared to a column that does not comprise crosslinked streptavidin-conjugated particles.

22.-43. (canceled)
